(19) United States
(12) Patent Application Publication
(10) Pub. No.: US 2023/0372290 A1
(43) Pub. Date: Nov. 23, 2023

(54) FORMULATIONS COMPRISING BOTANICAL EXTRACTS

(71) Applicant: APEX BIOTECH RESEARCH PTY LTD, Clayton, Victoria (AU)

(72) Inventors: Zhicheng XIAO, Clayton, Victoria (AU); Sa XIAO, Clayton, Victoria (AU); Gang PENG, Clayton, Victoria (AU); Zhiyong HE, Clayton, Victoria (AU)

(21) Appl. No.: 18/246,931

(22) PCT Filed: Sep. 28, 2021
(86) PCT No.: PCT/AU2021/051126

§ 371 Date: Mar. 28, 2023

(30) Foreign Application Priority Data
Sep. 28, 2020 (AU) 2020903493
Sep. 30, 2020 (AU) 2020903527
Apr. 21, 2021 (AU) 2021901178

(51) Int. Cl. A61K 31/365; A61K 31/7034; A61K 45/06; A61K 31/56; A61K 9/00; A61K 36/19; A61K 36/73; A61K 36/53; A61K 36/70; A61P 31/14

(52) U.S. Cl. CPC A61K 31/365; A61K 31/7034; A61K 45/06; A61K 31/56; A61K 9/0053; A61K 36/19; A61K 36/73; A61K 36/53; A61K 36/70; A61P 31/14

(57) ABSTRACT

The invention relates generally to combinations and compositions comprising botanical extracts useful in the treatment or prevention of viral and bacterial infections. In particular, the invention relates to combinations and compositions comprising two or more compounds selected from andrographolide or a derivative, or pharmaceutically acceptable salt thereof, ursolic acid, or a pharmaceutically acceptable salt thereof, and piceid or a derivative, or pharmaceutically acceptable salt thereof and use of the combinations and compositions in the treatment or prevention of viral and bacterial infections.

### FIELD OF THE INVENTION

The invention relates generally to combinations and compositions comprising botanical extracts useful in the treatment or prevention of viral and bacterial infections.

### BACKGROUND OF THE INVENTION

Coronaviruses are a large family of viruses that usually cause mild to moderate upper-respiratory tract illnesses, like the common cold. However, new coronaviruses have recently emerged from animal reservoirs that can cause serious and widespread illness and death. The severe acute respiratory syndrome coronavirus (SARS-CoV) and Middle East respiratory syndrome coronavirus (MERS-CoV) cause severe respiratory tract disease with high mortality rates.

The severe acute respiratory syndrome coronavirus 2 (SARS-CoV-2) is the causative agent of the global COVID-19 pandemic. SARS-CoV-2 was first reported in the Hubei province of China in late 2019. Following the SARS outbreak of 2002/2003 and the 2012 MERS epidemic, COVID-19 is the third notable outbreak in the 21^{st} century (Yang, T., et al. J. Autoimmun., 2020, 109, 102434; Da Costa, V. G. et al. Arch. Virol., 2020, 165(7), 1517-1526). As at 20 Sep. 2020, there are 30,369,778 confirmed cases globally and 948,795 confirmed deaths (https://covid19.who.int).

SARS-CoV, MERS-CoV and SARS-CoV-2 belong to the family of Coronaviridae and the genus Betacoronavirus. SARS-CoV-2 is a spherical, enveloped, single-stranded positive RNA virus that shares around 79.6% identity with the genome of SAR-CoV. The virions is composed of nucleic acid and nucleocapsid protein to form the helical nucleocapsid. The lipid envelope is studded with structural proteins including the membrane (M) glycoprotein, the envelope (E) protein and the spike (S) glycoprotein. Virus infection is initiated by interaction between S glycoprotein and host cell surface receptors. S glycoprotein is cleaved by cellular serine proteases TMPRSS2 into S1 and S2 subunits, which are responsible for receptor recognition and membrane fusion. Membrane fusion allows for release of the viral genome into the cellular cytoplasm, followed by RNA replication.

S glycoproteins have been observed in other viruses of the Coronaviridae family to mediate virus entry by contacting specific host-receptors located on the surface of cells. Host-guest recognition is virus-specific, with specificity being determined by virus tropism and pathogenesis (Ou, X., et al. Nat. Commun, 2020, 11(1), 1620; Walls, A. C., et al. Cell, 2020, 181(2), 281-292). Both SARS-CoV and SARS-CoV-2 enter host cells via the angiotensin-converting enzyme 2 (ACE2) receptor on the membrane of host cells. Membrane fusion of the virus and the host cell is activated after binding, and viral RNA is subsequently released into the cytoplasm, establishing infection (Hoffmann, M., et al. Cell, 2020, 181(2), 271-280).

Coronaviruses feature the largest known RNA virus genomes ranging approximately from 26 to 32 kb (Song, Z. et al. Viruses, 2019, 11(1) 59; Anand, K., et al. Science, 2003, 300(5626), 1763-1767), and contain at least 6 open reading frames (Chen, Y., et al. J Med Virol, 2020, 92(4), 418-423; Gordon, D. E. et al. Nature, 2020, 10.1038). The major reading frame, ORF 1ab, encodes for two overlapping polyproteins (pp1a and pp1ab), which are cleaved into 16 non-structural proteins by the main protease (M^{pro}) and the papain-like protease (PL^{pro}) (Ullrich, S. and Nitsche, C. Bioorganic & Medicinal Chemistry Letters, 2020, 30(17), 127377).

Currently there are few approved treatments for human coronaviruses or potentially lethal zoonotic coronaviruses such as SARS, MERS or COVID-19. Accordingly, there exists a need to develop potent and effective treatments for viral infections including coronavirus infections.

### SUMMARY OF THE INVENTION

New antiviral combinations, compositions and methods are provided for treating viral infections.

Accordingly, in one aspect the present invention provides an antiviral combination comprising two or more compounds selected from andrographolide or a derivative, or pharmaceutically acceptable salt thereof, ursolic acid, or a pharmaceutically acceptable salt thereof, and piceid or a derivative, or pharmaceutically acceptable salt thereof.

In another aspect, the invention provides a pharmaceutical composition comprising the antiviral combination according to the invention and at least one pharmaceutically acceptable carrier or diluent.

In one aspect, the invention provides pharmaceutical composition comprising an extract of Andrographis paniculata, together with ursolic acid, or a pharmaceutically acceptable salt thereof, and piceid, or a derivative, or pharmaceutically acceptable salt thereof.

In a further aspect, the present invention provides a method of treating or preventing a viral infection in a subject in need thereof comprising administering to the subject an effective amount of the antiviral combination or pharmaceutical composition according to the invention.

In another aspect, the present invention provides a method of treating or preventing a bacterial infection in a subject in need thereof comprising administering to the subject an effective amount of the antiviral combination or pharmaceutical composition according to the invention.

In yet another aspect, the present invention provides an antiviral combination or pharmaceutical composition according to the invention for use in the treatment or prevention of a viral infection.

In a further aspect, the present invention provides a combination or pharmaceutical composition according to the invention for use in the treatment or prevention of a bacterial infection.

These and other aspects of the present invention will become more apparent to the skilled addressee upon reading the following detailed description in connection with the accompanying examples and claims.

### BRIEF DESCRIPTION OF THE DRAWINGS

The invention will herein be described by way of example only with reference to the following non-limiting Figures in which:

FIG. 1 illustrates the effects of andrographolide (UP-A) piceid (UP-P) and ursolic acid (UP-U) on the expression of ACE2 mRNA in Vero (AGM) cells. Cells were incubated for 48 hours with the compounds. ACE2 expression is measured by quantitative reverse transcriptase RT-PCT. Data shows mean±SEM. * vs plate control p<0.05.

FIG. 2 illustrates the effects of andrographolide (UP-A) and piceid (UP-P) on the expression of ACE2 mRNA in Vero (AGM) cells and human lung epithelial (A549) cells. Cells were incubated for 24 hours with the compounds. ACE2 expression is measured by quantitative reverse transcriptase RT-PCT. Data shows mean±SEM. * vs plate control p<0.05.

FIG. 3 illustrates the expression of ACE2 protein by ELISA in cell lysate of CACO-2 cells 72 hours following treatment with andrographolide (UP-A) and piceid (UP-P) (1 μM) daily for three days. Data shows mean±SEM; * p<0.05.

### DETAILED DESCRIPTION OF THE INVENTION

Combination therapy is well established in the treatment of HIV infections and is emerging in the treatment of other viral infections due to drug resistance and as a means of combating viral infections that are otherwise difficult to treat. Treatment guidelines published by the United States Department of Health and Human Services provide that achievement of viral suppression requires the use of combination therapies, i.e., several drugs from at least two or more drug classes.

With this in mind, it is envisaged that combination therapy comprising certain botanical extracts will provide a novel treatment regimen for viral infections. Plant derived natural products play a crucial role in the development of new therapeutic agents. They have been suggested to combat viral infections by targeting viral receptors (Chang & Woo, 2003; Keyaerts, et al. 2007), viral integration (Kim et al. 2010), reverse transcription (Zhang et al. 2014) viral replication and viral protein translation (Mansouri et al. 2009). In particular, it has been found that combinations comprising two or more compounds selected from andrographolide or a derivative, or pharmaceutically acceptable salt thereof, ursolic acid, or a pharmaceutically acceptable salt thereof, and piceid, or a derivative, or pharmaceutically acceptable salt thereof have promising antiviral activity.

Accordingly, in one embodiment the present invention provides an antiviral combination comprising two or more compounds selected from andrographolide or a derivative, or pharmaceutically acceptable salt thereof, ursolic acid, or a pharmaceutically acceptable salt thereof, and piceid, or a derivative, or pharmaceutically acceptable salt thereof.

The term “combination”, as used herein refers to a composition or kit of parts where the combination partners as defined above can be dosed dependently or independently or by use of different fixed combinations with distinguished amounts of the combination partners, i.e., simultaneously or at different time points. The combination partners can then, e.g., be administered simultaneously or chronologically staggered, that is at different time points and with equal or different time intervals for any part of the kit of parts. The ratio of the total amounts of the combination partners to be administered in the combination can be varied, e.g. in order to cope with the needs of a patient sub-population to be treated or the needs of the single patient which different needs can be due to age, sex, body weight, etc. of the patients.

In one embodiment it is envisaged that the antiviral combination will comprise andrographolide or a derivative, or pharmaceutically acceptable salt thereof, and ursolic acid, or a pharmaceutically acceptable salt thereof. In another embodiment, the antiviral combination is envisaged to comprise andrographolide or a derivative, or pharmaceutically acceptable salt thereof, and piceid or a derivative, or pharmaceutically acceptable salt thereof. In a further embodiment, it is envisaged that the antiviral combination will comprise ursolic acid, or a pharmaceutically acceptable salt thereof, and piceid or a derivative, or pharmaceutically acceptable salt thereof. In yet another embodiment, the antiviral combination is envisaged to comprise andrographolide or a derivative, or pharmaceutically acceptable salt thereof, ursolic acid, or a pharmaceutically acceptable salt thereof, and piceid or a derivative, or pharmaceutically acceptable salt thereof.

Andrographolide, is a lactone diterpenoid of the following structure:

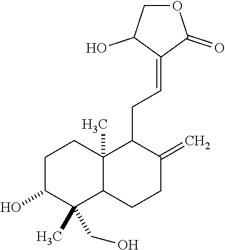 [SMILES: C=C1CCC2[C@@](C)(CC[C@@H](O)[C@@]2(C)CO)C1C/C=C1/C(=O)OCC1O]

### Andrographolide (3-[2-[Decahydro-6-hydroxy-5-(hydroxymethyl)-5,8a-dimethyl-2-methylene-1-napthalenyl]ethylidene]dihydro-4-hydroxy-2(3H)-furanone)

Andrographolide is highly abundant in the leaves of Andrographis paniculata, commonly known as creat or green chiretta, an herbaceous plant native to India and Sri Lanka. Andrographis paniculata is widely cultivated in Southern and Southeast Asia for its medicinal affects and both Andrographis paniculata extract and andrographolide have found various uses, for example, as anti-inflammatory, anti-tumour, and anti-hyperglycemia agents.

Favourably, andrographolide has been used extensively in traditional medicine in Asia and is considered safe to consume.

Ursolic acid is a pentacyclic triterpenoid compound with the following structure:

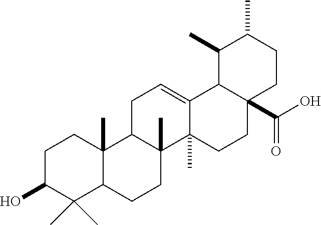

### Ursolic acid (1S,2R,4aS,6aR,6aS,6bR,8aR,10S,12aR,14bS)-10-Hydroxy-1,2,6a,6b,9,9,12a-heptamethyl-2,3,4,5,6,6a,7,8,8a,10,11,12,13,14b-tetradecahydro-1H-picene-4a-carboxylic acid)

It is found in many plants including Eriobotrya japonica, Cadamba, Mirabillis jalapa as well as in the waxy coating on apples and in many fruits and herbs such as rosemary, thyme, basil, bilberries, cranberries, elder flower, and peppermint.

Piceid (polydatin) is a stillbenoid glucoside and is a major resveratrol found in grape juices. Piceid can be found in the bark of Picea sitchensis and can be isolated from Reynoutria japonica. Piceid has the structure:

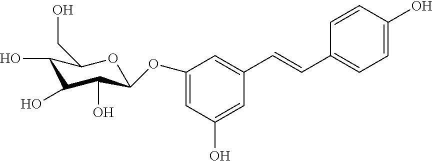

### Piceid (2-[3-Hydroxy-5-[(E)-2-(4-hydroxyphenyl)ethenyl]phenoxy]-6-(hydroxymethyl)oxane-3,4,5-triol)

Piceid and its derivative, resveratrol, have been shown to act as antioxidants and are the compounds linking the health benefits of red wine. Piceid and resveratrol are also suggested to have anti-proliferative and anti-inflammatory effects (Cheng, S. D. et al. PLoS ONE, 8(1): e54505).

Here, piceid has been identified as a compound to target M^{pro}. Initial in silico and enzymology studies indicate that piceid is a promising candidate targeting M^{pro}.

In one embodiment, the antiviral combinations and compositions comprise a derivative of andrographolide. As mentioned above, andrographolide is derived from the leaves of Andrographis paniculata. In addition to andrographolide, several derivatives have also been found to possess beneficial therapeutic qualities. Such derivatives include, but are not limited to:
14-deoxy-didehydroandrographolide;
14-dehydroxyandrographolide-12-sulfonic acid sodium salt;
14-alpha-lipoyl andrographolide;
andrographiside;
andrograpanin;
14-dehydroxy-11,12-didehydroandrographolide-3,19-bis(succinic acid) potassium salt;
14-dehydroxy-11,12-didehydroandrographolide-3,19-bis(succinic acid) potassium sodium salt;
(R)-((1R,5aS,6R,9aS)-1,5a-dimethyl-7-methylene-3-oxo-6-((E)-2-(2-oxo-2,5-dihydrofuran-3-yl)ethenyl)decahydro-1H-benzo [c]azapin-1-yl)methyl 2-amino-3-phenylpropanoate;
2-((1R,5R,6R,8aS)-6-hydroxy-5-(hydroxymethyl)-5,8a-dimethyl-2-methylenedecahydronaphthalen-1-yl)ethyl benzoate;
(1S,2R,4aS,5R,8aS)-1-formamido-1,4a-dimethyl-6-methylene-5-((E)-2-(2-oxo-2,5-dihydrofuran-3-yl)ethenyl)decahydronaphthalen-2-yl 5-((R)-1,2-pentanoate;
(1S,2R,4aS,5R,8aS)-1-formamido-1,4a-dimethyl-6-methylene-5-((E)-2-(2-oxo-2,5-dihydrofuran-3-yl)ethenyl)decahydronaphthalen-2-yl 2-nitrobenzoate; and
(S)-(1S,2R,4aS,5R,8aS)-1-formamido-1,4a-dimethyl-6-methylene-5-((E)-2-(2-oxo-2,5-dihydrofuran-3-yl)ethenyl)decahydronaphthalen-2-yl-2-amino-3-phenylpropanoate.

In another embodiment, the antiviral combinations and compositions comprise a derivative of ursolic acid. Such derivatives include resveratrol.

It will be understood that the compounds of the invention may exist in a plurality of equivalent tautomeric forms. For the sake of clarity the compounds have been depicted as single tautomers, despite all such tautomeric forms being considered within the scope of the invention.

It will be noted that the structures of some of the compounds of the invention include asymmetric carbon atoms. It is to be understood accordingly that the isomers arising from such asymmetry (e.g., all enantiomers, stereoisomers, rotamers, tautomers, diastereomers, or racemates) are included within the scope of this invention. The present invention includes within its scope all of these stereoisomeric forms either isolated (in, for example, enantiomeric isolation), or in combination (including racemic mixtures and diastereomic mixtures).

The invention thus also relates to compounds in substantially pure stereoisomeric form with respect to the asymmetric centres of the amino acid residues, e.g., greater than about 90% de, such as about 95% to 97% de, or greater than 99% de, as well as mixtures, including racemic mixtures, thereof. The skilled person will appreciate that there are a range of techniques available to produce achiral compounds of the invention in racemic, enantioenriched or enantiopure forms. As an example, enantioenriched or enantiopure forms of the compounds may be produced through stereoselective synthesis and/or through the use of chromatographic or selective recrystallisation techniques.

The compounds of the invention may be in crystalline form or as solvates (e.g. hydrates) and it is intended that both forms are within the scope of the present invention. The term “solvate” is a complex of variable stoichiometry formed by a solute (in this invention, a compound of the invention) and a solvent. Such solvents should preferably not interfere with the biological activity of the solute. Solvents may be, by way of example, water, acetone, ethanol or acetic acid. Methods of solvation are generally known within the art.

Where the compound comprises one or more functional groups that may be protonated or deprotonated (for example at physiological pH) the compound may be prepared or isolated as a pharmaceutically acceptable salt. It will be appreciated that the compound may be zwitterionic at a given pH. As used herein the expression “pharmaceutically acceptable salt” refers to the salt of a given compound, wherein the salt is suitable for administration as a pharmaceutical. Such salts may be prepared during chemical synthesis, for example, by the reaction of an acid or a base with an amine or a carboxylic acid group, respectively. Pharmaceutically acceptable salts of the compounds may also be isolated from herbal extracts.

Pharmaceutically acceptable acid addition salts may be prepared from inorganic and organic acids. Examples of inorganic acids include hydrochloric acid, hydrobromic acid, sulfuric acid, nitric acid, phosphoric acid and the like. Examples of organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like. For example, the nitrogen atom in the acyclic portion of the compounds of the invention may undergo reaction with an acid to form the acid addition salt.

Pharmaceutically acceptable base addition salts may be prepared from inorganic and organic bases. Corresponding counterions derived from inorganic bases include the sodium, potassium, lithium, ammonium, calcium and magnesium salts. Organic bases include primary, secondary and tertiary amines, substituted amines including naturally-occurring substituted amines, and cyclic amines, including isopropylamine, trimethyl amine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, and N-ethylpiperidine. For example, where the compound of the invention possesses a phosphonate group the compound may undergo reaction with a base to form the base addition salt.

Acid/base addition salts tend to be more soluble in aqueous solvents than the corresponding free acid/base forms.

The present invention also provides a pharmaceutical composition comprising the antiviral combination according to the invention, together with at least one pharmaceutically acceptable carrier or diluent. Accordingly, in one embodiment, the pharmaceutical composition will comprise andrographolide or a derivative, or pharmaceutically acceptable salt thereof, and ursolic acid, or a pharmaceutically acceptable salt thereof. In another embodiment, the pharmaceutical composition will comprise andrographolide or a derivative, or pharmaceutically acceptable salt thereof, and piceid or a derivative, or pharmaceutically acceptable salt thereof. In a further embodiment, the pharmaceutical composition will comprise ursolic acid, or a pharmaceutically acceptable salt thereof, and piceid or a derivative, or pharmaceutically acceptable salt thereof. In yet another embodiment, the pharmaceutical composition will comprise andrographolide or a derivative, or pharmaceutically acceptable salt thereof, ursolic acid, or a pharmaceutically acceptable salt thereof, and piceid or a derivative, or pharmaceutically acceptable salt thereof.

The term “composition” is intended to include the formulation of an active ingredient with encapsulating material as carrier, to give a capsule in which the active ingredient (with or without other carrier) is surrounded by carriers.

In another embodiment, the pharmaceutical composition will comprise two or more compounds selected from andrographolide or a derivative, or pharmaceutically acceptable salt thereof, ursolic acid, or a pharmaceutically acceptable salt thereof, and piceid, or a derivative, or pharmaceutically acceptable salt thereof and one or more additional therapeutic agents. In one embodiment, the one or more additional therapeutic agents are antiviral agents. Suitable antiviral agents are provided hereinabove.

In another embodiment, the antiviral combinations and compositions comprise one or more additional therapeutic agents. In one embodiment, the one or more additional therapeutic agents are antiviral agents.

The term “antiviral agent” means any currently known therapeutic compounds for the treatment of a viral infection. Suitable antiviral agents include, but are not limited to, remdesivir, dexamethasone, gimsilumab, sarilumab, tocilizumab, anakinra, ruxolitinib, baricitinib, fedratinib, chloroquine, hydroxychloroquine, lopinavir, ritinovir, favipiravir, EIDD-2801, baricitinib, methylprednisolone, heparin, zinc, arbidiol/umifenovir, darunavir, oseltamivir, emtricitibine, tenofovir, baloxavir marboxil, danoprevir, dipyridamole, fingolimod, losartan, azithromycin, ribavirin, triazavirin, tranilast, ebastine, quercetin, glycyrrhizin, baicalin, patchouli alcohol, luteolin, hesperidin, emodin, kaempferol, lignin, betulinic acid, tanshinone, cryptotanshinone, dihydrotanshinone, I, tanshinone IIA, curcumin, shikonon and matrine.

In a further embodiment there is provided a pharmaceutical composition comprising an extract of Andrographis paniculata, together with ursolic acid, or a pharmaceutically acceptable salt thereof, and piceid, or a derivative, or pharmaceutically acceptable salt thereof. As mentioned above, andrographolide is one of several compounds derived from Andrographis paniculata that has been found to possess beneficial therapeutic qualities including antiviral and anti-inflammatory activity.

In a further embodiment, the ursolic acid, or a pharmaceutically acceptable salt thereof, and/or piceid, or a derivative, or pharmaceutically acceptable salt thereof are also provided in the form of a plant extract. Accordingly, in one embodiment the invention provides a pharmaceutical composition comprising an extract of Andrographis paniculata, together with an extract of a plant selected from the group consisting of Eriobotrya japonica, rosemary and thyme, and/or an extract from Reynoutria japonica.

The antiviral combinations and compositions of the present invention may be used in the treatment and/or prevention of a range of viral infections. As used herein, treatment may include alleviating or ameliorating the symptoms, diseases or conditions associated with the viral infection being treated, including reducing the severity and/or frequency of the viral infection. As used herein, prevention may include preventing or delaying the onset of, inhibiting the progression of, or halting or reversing altogether the onset or progression of the particular symptoms, disease or condition associated with a viral infection.

Accordingly, in one embodiment the present invention provides a method of treating or preventing a viral infection in a subject in need thereof comprising administering to the subject an effective amount of an antiviral combination or a pharmaceutically composition according to the invention.

In one embodiment, the method will comprise administering to the subject an effective amount of andrographolide or a derivative, or pharmaceutically acceptable salt thereof, and ursolic acid, or a pharmaceutically acceptable salt thereof. In another embodiment, the method will comprise administering to the subject an effective amount of andrographolide or a derivative, or pharmaceutically acceptable salt thereof, and piceid or a derivative, or pharmaceutically acceptable salt thereof. In a further embodiment, the method will comprise administering to the subject an effective amount of ursolic acid, or a pharmaceutically acceptable salt thereof, and piceid or a derivative, or pharmaceutically acceptable salt thereof. In yet another embodiment, the method will comprise administering to the subject an effective amount of andrographolide or a derivative, or pharmaceutically acceptable salt thereof, ursolic acid, or a pharmaceutically acceptable salt thereof, and piceid or a derivative, or pharmaceutically acceptable salt thereof.

In another embodiment, the method will comprise administering to the subject an effective amount of two or more compounds selected from andrographolide or a derivative, or pharmaceutically acceptable salt thereof, ursolic acid, or a pharmaceutically acceptable salt thereof, and piceid, or a derivative, or pharmaceutically acceptable salt thereof and one or more additional therapeutic agents. In one embodiment, the one or more additional therapeutic agents are antiviral agents. Suitable antiviral agents are provided hereinabove.

The present invention further provides an antiviral combination or a pharmaceutically composition according to the invention for use in the treatment or prevention of a viral infection.

In another embodiment the invention provides use of two or more of andrographolide or a derivative, or pharmaceutically acceptable salt thereof, ursolic acid, or a pharmaceutically acceptable salt thereof, and piceid or a derivative, or pharmaceutically acceptable salt thereof, in the manufacture of a medicament for the treatment or prevention of a viral infection.

The term “viral” refers to any virus that is known to cause infection in a host. In one embodiment, the virus is selected from the group consisting of a picornavirus, a coronavirus, an influenza virus, a parainfluenza virus, a respiratory syncytial virus, an adenovirus, an enterovirus, and a metapneumovirus. Suitable antigens of picornavirus, a coronavirus, an influenza virus, a parainfluenza virus, a respiratory syncytial virus, an adenovirus, an enterovirus, and a metapneumovirus will be familiar to persons skilled in the art.

In an embodiment, the virus is of the family Coronaviridae. The Coronaviridae family is typically divided into Coronavirinae and Torovirinae sub-families, which are further divided into six genera: Alphacoronavirus, Betacoronavirus, Gammacoronavirus, Deltacoronavirus, Torovirus, and Bafinivirus. While viruses in the genera Alphacoronaviruses and Betacoronaviruses infect mostly mammals, the Gammacoronavirus infect avian species and members of the Deltacoronavirus genus have been found in both mammalian and avian hosts (Phan et al., Virus Evol. 2018; 4(2): vey035).

Suitable viruses of the family Coronaviridae will be familiar to persons skilled in the art, illustrative examples of which include Alphaletovirus (see, e.g., Bukhari et al.; Virology. 2018; 524:160-171) and Coronavirus (see, e.g., Fehr and Perlman; Coronaviruses. 2015; 1282: 1-23). Thus, in an embodiment disclosed herein, the virus is selected from the group consisting of Alphaletovirus and Coronavirus. In an embodiment, the virus is a coronavirus. In an embodiment disclosed herein, the coronavirus is selected from the group consisting of Alphacoronavirus, Betacoronavirus, Deltacoronavirus and Gammacoronavirus. In an embodiment, the coronavirus is Betacoronavirus. Suitable Betacoronaviruses will be familiar to persons skilled in the art, an illustrative example of which includes a Sarbecovirus. In an embodiment, the Betacoronavirus is a Sarbecovirus. Suitable Sarbecoviruses will be familiar to persons skilled in the art, illustrative examples of which include Severe acute respiratory syndrome-related coronavirus, Severe acute respiratory syndrome coronavirus (SARS-CoV) and Severe acute respiratory syndrome coronavirus 2 (SARS-CoV-2). In an embodiment, the Sarbecovirus is selected from the group consisting of severe acute respiratory syndrome-related coronaviruses SARS-CoV and SARS-CoV-2. In an embodiment, the Sarbecovirus is SARS-CoV-2. In an embodiment, the SARS-CoV-2 is encoded by a nucleic acid sequence of NCBI Accession Number NC_045512.

In one embodiment, the viral infection is caused by a virus selected from the group consisting of a picornavirus, a coronavirus, an influenza virus, a parainfluenza virus, a respiratory syncytial virus, an adenovirus, an enterovirus, and a metapneumovirus. In a preferred embodiment, the virus is a coronavirus. In a preferred embodiment, the coronavirus is SARS-CoV-2.

In addition to treating, attenuating or preventing a viral infection, combinations of andrographolide, ursolic acid and piceid, as described herein, can be beneficial for the treatment or prevention of microbial and bacterial infection, including secondary microbial or bacterial infection. For example, a major consequence of disease progression with patients suffering from severe acute respiratory syndrome is secondary bacterial infections. At least one in seven COVID-19 patients were found to be additionally infected with a secondary bacterial infection, with 50% of the fatalities during the SARS-CoV-2 epidemic in Wuhan caused by untreated or untreatable bacterial infections, in most cases in the lung (Zhou, F. et al. Lancet, 2020, 395, 1054-1062).

Accordingly, in one embodiment there is provided a method of treating or preventing a bacterial infection in a subject in need thereof comprising administering to the subject an effective amount of the antiviral combination or pharmaceutical composition according to the invention. In an embodiment, the bacterial infection is a secondary infection to a viral infection.

In another embodiment disclosed herein, there is provided use of two or more of andrographolide or a derivative, or pharmaceutically acceptable salt thereof, ursolic acid, or a pharmaceutically acceptable salt thereof, and piceid or a derivative, or pharmaceutically acceptable salt thereof, in the manufacture of a medicament for the treatment or prevention of a bacterial infection. In an embodiment, the bacterial infection is a secondary infection to a viral infection.

Secondary bacterial infection typically develops in patients during or after initial infection with an infective pathogen and are associated with high morbidity and mortality rates (Mallia, P., et al. Am. J. Respir. Crit. Care Med. 2012, 186, 1117-1124). Around 50 million deaths were ascribed to bacterial co-infections during the 1918-1919 Spanish Flu pandemic (Kash, J. C., and Taubenberger, J. K. Am. J. Pathol. 2015, 185, 1528-1536). Secondary bacterial infections are facilitated by a compromised immune system as a result of the primary infection that is unable to appropriately respond to both pathogen types

The terms “microbial”, includes any microscopic organism or taxonomically related macroscopic organism within the categories algae, bacteria, fungi and protozoa or the like. The bacterial infection may be caused by one or more species selected from one or more of the Gram-negative bacterial genera: Acinetobacter; Actinobacillus; Bartonella; Bordetella; Brucella; Burkholderia; Campylobacter; Cyanobacteria; Enterobacter; Erwinia; Escherichia; Francisella; Helicobacter; Hemophilus; Klebsiella; Legionella; Moraxella; Morganella; Mycobacterium; Neisseria; Pasteurella; Proteus; Providencia; Pseudomonas; Salmonella; Serratia; Shigella; Stenotrophomonas; Treponema; Vibrio; and Yersinia. Specific examples include, but are not limited to, infections caused by Helicobacter pylori and uropathogenic Escherichia coli.

The bacterial infection may be caused by one or more species selected from one or more of the Gram-positive bacterial genera: Actinobacteria; Bacillus; Clostridium; Corynebacterium; Enterococcus; Listeria; Nocardia; Staphylococcus; and Streptococcus.

Protozoal infections include, but are not limited to, infections caused by Leishmania, Toxoplasma, Plasmodia (which are understood to be the causative agent(s) of malarial infection), Theileria, Anaplasma, Giardia, Tritrichomonas, Trypanosoma, Schistosoma, Coccidia, and Babesia. Specific examples include Plasmodium falciparum, Plasmodium vivax, Plasmodium malariae, Plasmodium knowlesi, Plasmodium ovale and Giardia lamblia.

The term “subject” is intended to include organisms such as mammals, e.g. humans, dogs, cows, horses, pigs, sheep, goats, cats, mice, rabbits, rats, and transgenic non-human animals. In certain embodiments, the subject is a human, e.g. a human suffering from, at risk of suffering from, or potentially capable of suffering from a microbial infection. In another embodiment, the subject is a cell.

“Administering” or “administration” refers to the delivery of two or more therapeutic compounds to a subject or patient. In one embodiment, the administration is co-administration such that two or more therapeutic compounds are delivered together during the course of the treatment. In one embodiment, two or more therapeutic compounds may be co-formulated into a single dosage form or “combined dosage unit”, or formulated separately and subsequently combined into a combined dosage unit, as is typically for oral administration as a mono or bilayer tablet or capsule.

In one embodiment, two or more compounds selected from andrographolide or a derivative, or pharmaceutically acceptable salt thereof, ursolic acid, or a pharmaceutically acceptable salt thereof, and piceid, or a derivative, or pharmaceutically acceptable salt thereof are administered to a human patient in need thereof in an effective amount of each compound, independently from about 0.1 mg to about 1000 mg per compound per day. In one embodiment, the effective amount of the combination treatment is independently from about 0.1 mg to about 500 mg per compound per day. In one embodiment, the effective amount of the combination treatment is independently from about 0.5 mg to about 200 mg per compound per day. In one embodiment, the effective amount of the combination treatment is independently from about 1 mg to about 100 mg per compound per day. In other embodiments, the effective amount of the combination treatment is for each component, about 1 mg, about 3 mg, about 5 mg, about 10 mg, about 15 mg, about 18 mg, about 20 mg, about 30 mg, about 40 mg, about 60 mg, about 80 mg, about 100 mg, about 200 mg, or about 500 mg each per day.

Co-administration may also include administering component drugs e.g., two or more compounds selected from andrographolide or a derivative, or pharmaceutically acceptable salt thereof, ursolic acid, or a pharmaceutically acceptable salt thereof, and piceid, or a derivative, or pharmaceutically acceptable salt thereof. Such combination of two or more compounds selected from andrographolide or a derivative, or pharmaceutically acceptable salt thereof, ursolic acid, or a pharmaceutically acceptable salt thereof, and piceid, or a derivative, or pharmaceutically acceptable salt thereof may be administered simultaneously or in sequence (one after the other) within a reasonable period of time of each administration (e.g., about 1 minute to 24 hours) depending on the pharmacokinetic and/or pharmacodynamics properties of each agent or the combination. Co-administration may also involve treatment with a fixed combination wherein agents of the treatment regimen are combinable in a fixed dosage or combined dosage medium e.g., solid, liquid or aerosol. In one embodiment, a kit may be used to administer the drug or drug components.

The antiviral combinations and compositions of the invention are primarily intended for oral administration to enable delivery of the active compounds to the sight or infection, i.e. the respiratory system. Those skilled in the art may readily determine appropriate formulations for the compounds of the present invention using conventional approaches.

While it is possible for the active compounds to be administered alone it may be preferable to present them as pharmaceutical formulations. The formulations, both for veterinary and for human use, comprise at least two active compounds, as herein defined, together with one or more acceptable carriers and optionally other therapeutic ingredients, particularly those additional therapeutic ingredients as discussed herein. The carrier(s) must be “acceptable” in the sense of being compatible with the other ingredients of the formulation and physiologically innocuous to the recipient thereof.

All formulations will optionally contain excipients such as those set forth in the “Handbook of Pharmaceutical Excipients” (1986). Excipients include ascorbic acid and other antioxidants, chelating agents such as EDTA, carbohydrates such as dextran, hydroxyalkylcellulose, hydroxyalkylmethylcellulose, stearic acid and the like. The pH of the formulations ranges from about 3 to about 11, but is ordinarily about 7 to 10.

The formulations may conveniently be presented in unit dosage form and may be prepared by any of the methods well known in the art of pharmacy. Techniques and formulations generally are found in Remington's Pharmaceutical Sciences (Mack Publishing Co., Easton, PA). Such methods include the step of bringing into association the active ingredient(s) with the carrier which constitutes one or more accessory ingredients. In general, the formulations are prepared by uniformly and intimately bringing into association the active ingredient(s) with liquid carriers or finely divided solid carriers or both, and then, if necessary, shaping the product.

Formulations suitable for oral administration may be presented as discrete units such as capsules, cachets or tablets each containing a predetermined amount of the active ingredient(s); as a powder or granules; as a solution or a suspension in an aqueous or non-aqueous liquid; or as an oil-in-water liquid emulsion or a water-in-oil liquid emulsion. The active ingredient(s) may also be administered as a bolus, electuary or paste.

The compounds of the present invention may be administered by inhalation in the form of an aerosol spray from a pressurised dispenser or container, which contains a propellant such as carbon dioxide gas, dichlorodifluoromethane, nitrogen, propane or other suitable gas or combination of gases. The compounds may also be administered using a nebuliser.

Aqueous suspensions contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more colouring agents, one or more flavouring agents and one or more sweetening agents, such as sucrose or saccharin.

Dispersible powders and granules suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavouring and colouring agents, may also be present.

Oil suspensions may be formulated by suspending the active ingredient(s) in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavouring agents may be added to provide a palatable oral preparation. These compositions may be preserved by the addition of an antioxidant such as ascorbic acid.

The pharmaceutical compositions may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavouring agents. Syrups and elixirs may be formulated with sweetening agents, such as glycerol, sorbitol or sucrose. Such formulations may also contain a demulcent, a preservative, a flavouring or a colouring agent.

A tablet is made by compression or molding, optionally with one or more accessory ingredients. Compressed tablets may be prepared by compressing in a suitable machine the active ingredient(s) in a free-flowing form such as a powder or granules, optionally mixed with a binder, lubricant, inert diluent, preservative, surface active or dispersing agent. Moulded tablets may be made by molding in a suitable machine a mixture of the powdered active ingredient(s) moistened with an inert liquid diluent. The tablets may optionally be coated or scored and optionally are formulated so as to provide slow or controlled release of the active ingredient therefrom.

Tablets containing the active ingredient(s) in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatine or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period, for example, a time delay material such as glyceryl monostearate or glyceryl distearate alone or with a wax may be employed. The tablet may be a chewable tablet.

If desirable, the antiviral combinations and compositions according to the invention may be prepared in parenteral dosage forms, including those suitable for intravenous, intrathecal or epidural delivery. The pharmaceutical forms suitable for injectable use include sterile injectable solutions or dispersions, and sterile powders for the extemporaneous preparation of sterile injectable solutions. They should be stable under the conditions of manufacture and storage and may be preserved against reduction or oxidation and the contaminating action of microorganisms such as bacteria or fungi.

The solvent or dispersion medium for the injectable solution or dispersion may contain any of the conventional solvent or carrier systems for the compound, and may contain, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about where necessary by the inclusion of various antibacterial and antifungal agents, for example, parabens, chlorobutanol, phenol, sorbic acid, thimerosal and the like. In many cases, it will be preferable to include agents to adjust osmolarity, for example, sugars or sodium chloride. Preferably, the formulation for injection will be isotonic with blood. Prolonged absorption of the injectable compositions can be brought about by the use in the compositions of agents delaying absorption, for example, aluminium monostearate and gelatin. Pharmaceutical forms suitable for injectable use may be delivered by any appropriate route including intravenous, intramuscular, intracerebral, intrathecal, epidural injection or infusion.

Enteral formulations may be prepared in the form of suppositories by mixing with appropriate bases, such as emulsifying bases or water-soluble bases. It is also possible, but not necessary, for the compounds of the present invention to be administered topically, intranasally, intravaginally, intraocularly and the like.

It is especially advantageous to formulate the compositions in dosage unit form for ease of administration and uniformity of dosage. Dosage unit form as used herein refers to physically discrete units suited as unitary dosages for the subjects to be treated; each unit containing a predetermined quantity of active material calculated to produce the desired therapeutic effect in association with the required pharmaceutically acceptable vehicle. The specification for the novel dosage unit forms of the invention are dictated by and directly dependent on (a) the unique characteristics of the active material and the particular therapeutic effect to be achieved, and (b) the limitations inherent in the art of compounding active materials for the treatment of disease in living subjects having a diseased condition in which bodily health is impaired as herein disclosed in detail.

As mentioned above the principal active ingredients may be compounded for convenient and effective administration in therapeutically effective amounts with a suitable pharmaceutically acceptable vehicle in dosage unit form. A unit dosage form can, for example, contain the one or more of the active compounds in amounts ranging from 0.25 μg to about 200 mg. Expressed in proportions, the active compounds may be present in concentrations ranging from about 0.25 μg to about 200 mg/mL of carrier. In the case of compositions containing supplementary active ingredients, the dosages are determined by reference to the usual dose and manner of administration of the said ingredients.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The invention will now be described with reference to some specific examples and drawings. However, it is to be understood that the particularity of the following description is not to supersede the generality of the invention as hereinbefore described.

### Example 1: In Vitro Assessment of Antiviral Activity

Methods (adapted from Tilmanis et al. Antiviral Therapy 2017, 147, 142-8; Dowall et al. Viruses, 2016)

### Cells and Virus:

SARS-CoV-2 is grown in Vero cells, aliquoted and stored at −80° C. Antiviral activity of selected compounds is assessed in 96-well plates with Vero cell monolayers with a standard virus concentration of 100 TCID50/well or other specified moi.

### Stage 1A—Antiviral Activity

### Test Compounds:

Stock solutions of test compounds are prepared, aliquoted and frozen at −20° C. For testing, an aliquot is thawed and serial two-fold dilutions are prepared from 40 μM to 0.078 μM to achieve final concentrations of 20 μM to 0.039 μM after addition to an equal volume of media in the well.

Several compounds are required to be prepared in dimethyl sulfoxide (DMSO) solution. The highest concentration of DMSO does not exceed 0.05% when applied to the cells.

### Antiviral Assay:

### Screening Assay:

In the PC3 1ab media is removed from the wells of 96-well plates.

### Method:

SARS-CoV-2 is added at 100 TCID50 (tissue culture infectious dose 50 percent) in 50 μl to triplicate wells. Other wells are mock-infected (50 μl medium) and one set of wells receives no compound and no virus (50 μl medium).

After 1 h adsorption at room temperature, the inoculum is removed, wells washed ×1 with PBS and 100 μl of medium is added to each well. The ‘no compound no virus’ cells receive an additional 100 μl of medium.

100 μl of diluted compounds are added to relevant wells at concentrations of 40 μM to 0.078 μM; three wells with virus and mock-infected, leaving the ‘no compound no virus’ cells. The plates are incubated at 37° C. for 3 days.

Cells are microscopically assessed for SARS-CoV-2-induced cytopathic effect on day 3 post-infection. After cpe is recorded the supernatant from the virus wells is pooled: 140 μl is used for RNA extraction and RT-PCR for viral genome quantification, 100 μl is set aside for virus titration on 96 well plates of Vero cells (fresh or frozen) and the remaining 300+μl is frozen at −80 C until TCID50 is performed.

### Confirmatory Assay:

In the PC3 1ab media is removed from the inner wells of 96-well plates. Due to edge-effects, the outer wells are left with media added. This leaves 6 wells across and 10 wells lengthwise per plate.

### Method:

SARS-CoV-2 is added at 100 TCID50 (tissue culture infectious dose 50 percent) in 50 μl to triplicate wells. Other wells are mock-infected (50 μl medium) and one set of wells receives no drug and no virus (50 μl medium).

After 1 h adsorption at room temperature, the inoculum is removed, wells washed ×1 with PBS and 100 μl of medium is added to each well. The ‘no compound no virus’ cells receive an additional 100 μl of medium.

100 μl of diluted compounds at concentrations ranging from 40 μM to 2.5 μM are added to replicates per dilution; three wells with virus and mock-infected, leaving the ‘no compound no virus’ cells. The plates are incubated at 37° C. for 3 days.

Cells are microscopically assessed for SARS-CoV-2-induced cytopathic effect on day 3 post-infection. 140 μl of supernatant from each virus infected well and 1 mock infected well is harvested for RNA extraction and RT-PCR for viral genome quantification. The remaining volume from each virus infected well and 1 mock infected well is titered (fresh or frozen) on Vero cells in 96 well plates.

### Stage 1B—Cytotoxicity Testing

Compounds that show antiviral activity at concentrations <20 μM are evaluated for cytotoxicity. Cytotoxicity is assessed using the CellTiterGlo cell viability assay (CTG; Promega, USA), and the MTT (3-(4,5-di-methylthiazol-2-yl)-2,5-diphenyltetrazolium bromide; MP Biomedicals, USA) assay, following incubation of plates for 3 days at 37° C.

The CTG assay is performed as per manufacturer's instructions, and luminescence is measured using a FLUOstar Optima luminometer (BMG Labtech, Germany).

The procedure for the MTT assay is based on previously described methods (Mosmann, 1983). Briefly, cell monolayers are incubated for 4 h in the presence of 1 mg/mL MTT at 37° C. The supernatant is removed and 200 μl isopropanol (Sigma, USA) added to dissolve the purple formazan that is produced. Absorbance is measured at 570 nm using a Multiskan Ascent plate reader (Thermo Fisher, USA).

The 50% cytotoxic concentration for the compound (CC₅₀; compound concentration that reduces the cell viability by 50% compared to the cell only control) is determined using nonlinear regression analysis (GraphPad Prism, USA) for both cytotoxicity readouts.

### Stage 2—Antiviral Activity, Confirmatory Assay

### Transwells

### Confirmatory Assay:

Compounds that show promising results in the Vero cell screen are moved to confirmatory screening using normal human bronchial epithelial (NHBE) cells grown at the air-liquid interface (ALI).

NHBE cells are differentiated at ALI for 6-8 weeks in the tissue culture laboratory at ACDP.

### Method:

In the PC4 1ab NHBE cells in 24 well transwell plates are inoculated with SARS-CoV-2 at an MOI of 0.01 in triplicate wells.

After 1 h adsorption at room temperature, the inoculum is removed, wells washed ×1 with PBS.

100 μl of diluted compounds are added to relevant wells at three different concentrations based on the results from the Vero cell screen. The plates are incubated at 37° C. for 3 days.

Cells are microscopically assessed for SARS-CoV-2-induced cytopathic effect on day 3 post-infection. 140 μl of supernatant from each virus infected well is harvested for RNA extraction and RT-PCR for viral genome quantification. A further 50 ul is used to assess cytotoxicity using the Cytotox 96 Non-Radioactive Cytotoxicity (Promega). The remaining volume from each virus infected well is titered (fresh or frozen) on Vero cells in 96 well plates for TCID50.

### Example 2: Animal Study

### Study:

Using a ferret model of SARS-CoV-2, this Work assesses the efficacy of an antiviral candidate across two studies. The ferrets are initially housed at the Werribee animal facility where they will receive a prime and boost of the canine distemper vaccine (CDV) as a part of standard animal husbandry requirements with a washout period greater than six-weeks prior to enrolment. Ferrets are then transferred to the Institution's ACDP national facility and acclimatised for one week prior to administration of the SARS-CoV-2 vaccine candidate. Each study consists of 8 gender-balanced ferrets in a test group. Each study has two male and two female controls that receive saline. Before virus challenge, all ferrets are pre-sampled for baseline haematology, clinical chemistry and serology. In addition, rectal and oral swabs and a nasal wash are collected before ferrets are challenged intranasally with SARS-CoV-2 (10\hat{ }5 TCID 50). Ferrets are monitored for clinical signs, and rectal and oral swabs and nasal washes collected every 2-3 days for two weeks post challenge for virological assessment. Ferrets are monitored for fever, lethargy and restlessness, reduced interested in interactions with other ferrets/handlers, diarrhoea as well as any signs of respiratory disease which may include sneezing, coughing, nasal discharge, increased respiratory effort or laboured breathing. Animals are humanely killed at the first observation of any moderate or severe clinical signs or at the conclusion of the study (Day 21). A terminal blood sample is collected and a panel of tissues are collected at necropsy and assessed for the presence or absence of SARS-CoV-2.

### Example 3: Formulations

Representative formulations of the invention are provided below:

### KBD Spray:

Eriobotrya japonica extract
Equivalent to Eriobotrya japonica (Dry leaf) 12 mg/mL
Propolis 200 mg/mL
Reynoutria japonica extract
Equivalent to Reynoutria japonica (Dry root) 250 mg/mL

### A+KBD Spray:

Eriobotrya japonica extract
Equivalent to Eriobotrya japonica (Dry leaf) 34.5 mg/mL
Propolis 100 mg/mL
Reynoutria japonica extract
Equivalent to Reynoutria japonica (Dry root) 56 mg/mL
Andrographis paniculata extract
Equivalent to Andrographis paniculata (Dry) 2.8 mg/ml
Menthol 2 mg/ml

### KBD Chewable Tablets:

Reynoutria japonica extract
Equivalent to Reynoutria japonica (Dry root) 25 g
Eriobotrya japonica extract
Equivalent to Eriobotrya japonica (Dry leaf) 4.5 g
Echinacea purpurea extract
Equivalent to Echinacea purpurea (Dry herb) 840 mg
Propolis 250 mg

### KBD Hardgel Capsules:

Echinacea purpurea extract
Equivalent to Echinacea purpurea (Dry herb) 840 mg
Reynoutria japonica extract
Equivalent to Reynoutria japonica (Dry root) 3 g
Andrographis paniculata extract
Equivalent to Andrographis paniculata (Dry herb) 1.63 g
Eriobotrya japonica extract
Equivalent to Eriobotrya japonica (Dry leaf) 2.25 g
Propolis 200 mg
Vitamin D3 5 mcg (200 IU)

### Example 4: Antiviral Activity in Vero Cells

### Method:

Vero cells in 24 well plates were infected with 1000 TCID₅₀ (0.005MOI) of SARS-CoV-2 (Vic 01) in a volume of 100 μl for 1 hour at room temperature. Inoculum was removed and replaced with 500 μl of serially diluted compound(s) alone or in combination and incubated for 3 days at 37° C. and 5% CO₂. Controls included a cell control which received neither virus nor drug and vehicle control which received vehicle at the same concentration as the highest concentration of the drug tested. NHC (Beta-D-N⁴-hydroxycytidine) was used as positive control in the assay. On day 3, the wells were examined by microscopy for the presence of cytopathic effect (CPE). Supernatants from duplicate wells were harvested, pooled and stored at −80° C. till samples were titred for infectious virus on Vero cells in 96 well format and for viral RNA extraction. RT-PCR was performed on the extracted RNA samples using SARS-CoV-2 E gene-specific primers. GraphPad Prism was used for generation of graphs and for calculating IC₅₀ values from TCID₅₀ data.

### Results:

TABLE 1
Microscopy at day 3

| | | Androg- | Ursolic | | | |
|---|---|---|---|---|---|---|
| Drug conc. | Piceid | raphis | acid | | | P + |
| (μM) | (P) | (A) | (U) | P + A | P + U | A + U |
| 40 | CPE | Toxicity | Toxicity | Toxicity | Toxicity | Toxicity |
| | | or CPE | or CPE | or CPE | or CPE | or CPE |
| 20 | CPE | CPE | CPE | CPE | CPE | CPE |
| 10 | CPE | CPE | CPE | CPE | CPE | CPE |
| 5 | CPE | CPE | CPE | CPE | CPE | CPE |
| 2.5 | CPE | CPE | CPE | CPE | CPE | CPE |
| 1.25 | CPE | CPE | CPE | CPE | CPE | CPE |
| 0.625 | CPE | CPE | CPE | CPE | CPE | CPE |
| 0.3125 | CPE | CPE | CPE | CPE | CPE | CPE |
| 0.15625 | CPE | CPE | CPE | CPE | CPE | CPE |
| 0.078125 | CPE | CPE | CPE | CPE | CPE | CPE |
| Vehicle | — | — | — | — | — | — |

Data from the virus titration was graphed using GraphPad prism and an IC₅₀ of 3.251 μM was calculated for andrographis alone. The calculated IC₅₀ was reduced to 2.475 μM for the combination of andrographis and piceid and further reduced to 1.406 μM for the combination of andrographis, piceid and ursolic acid. Accordingly, the compound andrographis showed antiviral activity against SARS-CoV-2 alone and in combination with piceid and ursolic acid.

### Example 5: Effects of the Compounds on ACE2 Expression

### Method:

In-vitro testing was undertaken using human colonic epithelial cells (Caco-2) cells cultured in MCDB 131 medium (10% FCS with 10 mM glutamine, EGF and hydrocortisone) and African green monkey (Chlorocebus sp) derived VeroE6 cells cultured in aMEM medium. These cell-lines have endogenous high-surface expression of ACE2 and can be infected by SARS-CoV in-vitro. Cells were grown in 24-well plates to 80% confluence and then treated with fresh media containing andrographalide, piceid and ursolic acid (0-10 μM) in replicates of 8. After 1-3 days, cells were harvested by scraping in Trizol for RNA extraction. cDNA was synthesised from extracted RNA and gene expression of ACE2 mRNA was estimated by quantitative real-time RT-PCR using the TaqMan system based on real-time detection of accumulated fluorescence (ABI Prism 7700, Perkin-Elmer Inc). Gene expression was normalized to 18S mRNA and reported as fold change compared to the level of expression in untreated samples, which were given an arbitrary value of 1.0.

For ACE2 protein measurements, Caco-2 cells were scraped from each well in ice-cold RIPA buffer for ELISA assays. To determine the quantity of ACE2 in Caco-2 cells an Angiotensin Converting Enzyme 2 (ACE2) ELISA Kit (CusaBio, Wuhan, Hubei, China, catalogue number: CSB-E17204m) was performed on samples as per the manufacturers protocol. Briefly, each of the samples was loaded with Sample Diluent in duplicate wells. A standard curve was prepared from a Standard solution provided. 0, 6.25, 12.5, 25, 50, 100, 200 and 400 μg/mL of the standard was added to a series of wells in duplicate. The final volume of each standard well was made up to 250 mL with the addition of Sample Diluent. 100 mL of provided Biotin-antibody (1×) was added to each well and then after washing steps, 100 mL of HRP-avidin antibody (1×). The plate was put on shake mode on a CLARIOstarPlus Multi-Mode Microplate Reader (BMG Labtech, Ortenberg, Germany) to ensure proper mixing before being scanned at Ex/Em=570/540 nm in an end point mode.

### Results:

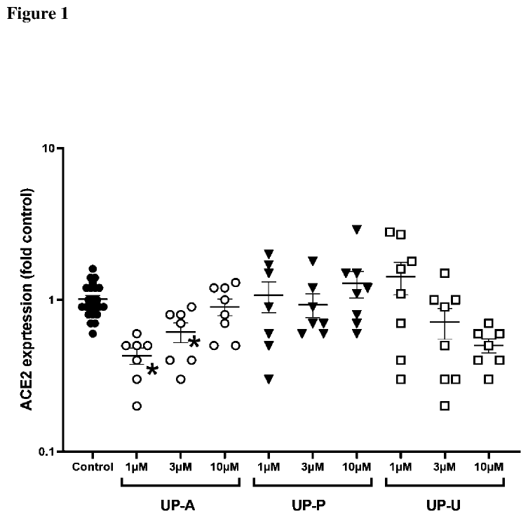

As can be seen from FIG. 1, andrographalide (UP-A) significantly decreased ACE2 mRNA expression in Vero (AGM) cells at 48 h, with the greatest effect seen at the lowest dose of 1 μM. Ursolic acid (UP-U) decreased ACE2 mRNA expression to a lesser extent in a dose dependent manner. Piceid had no discernible effect on ACE2 mRNA expression.

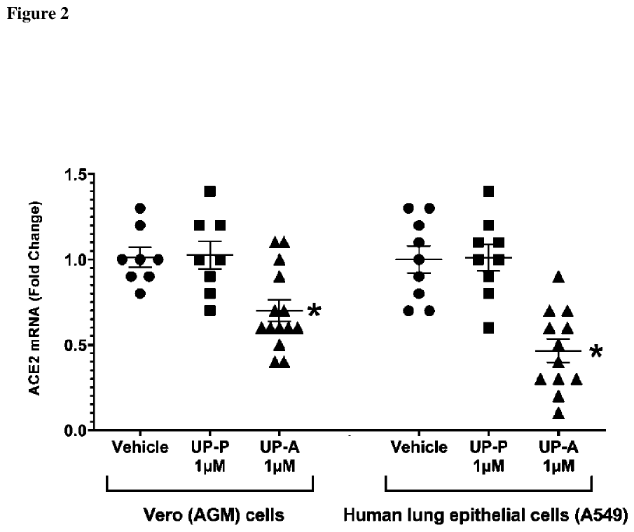

FIG. 2 verifies the effectiveness of andrographalide (UP-A) in decreasing ACE2 mRNA expression and illustrates that a similar effect is observed in human lung epithelial (A549) cells. The maximal effect was seen at 24 hours, decreasing at 48 hours and diminishing at 72 hours. This suggests that multiple dosing may be required, which is not unexpected for compounds that may be metabolised. The dose response suggests that 1 μM andrographalide achieves the maximal response, with a loss of efficacy observed below 0.5 μM and no substantial increase in activity at higher doses.

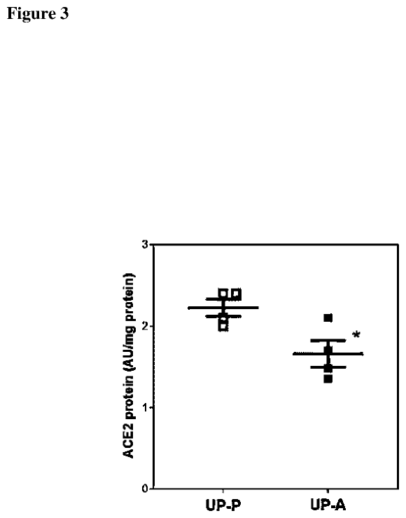

The reduction in ACE2 gene expression was consistent with the data shown in FIG. 3 that ACE2 protein levels, as measured by ELISA, decreased in CACO-2 cells 72 hours after treatment with 1 μM of andrographalide (UP-A) when compared to UP-P. These data suggest that the antiviral activity of UP-A is, at least in part, attributed to a reduction in the cell surface expression of ACE2, noting that ACE2 is the putative receptor for the receptor binding domain (RBD) of SARS-CoV-2.

1. An antiviral combination comprising two or more compounds selected from andrographolide or a derivative, or pharmaceutically acceptable salt thereof, ursolic acid, or a pharmaceutically acceptable salt thereof, and piceid, or a derivative, or pharmaceutically acceptable salt thereof.

2. The antiviral combination according to claim 1, comprising andrographolide or a derivative, or pharmaceutically acceptable salt thereof, and ursolic acid, or a pharmaceutically acceptable salt thereof.

3. The antiviral combination according to claim 1, comprising andrographolide or a derivative, or pharmaceutically acceptable salt thereof, and piceid or a derivative, or pharmaceutically acceptable salt thereof.

4. The antiviral combination according to claim 1, comprising ursolic acid, or a pharmaceutically acceptable salt thereof, and piceid or a derivative, or pharmaceutically acceptable salt thereof.

5. The antiviral combination according to claim 1, comprising andrographolide or a derivative, or pharmaceutically acceptable salt thereof, ursolic acid, or a pharmaceutically acceptable salt thereof, and piceid or a derivative, or pharmaceutically acceptable salt thereof.

6. The antiviral combination according to claim 1, wherein the derivative of andrographolide is selected from the group consisting of:
14-deoxy-didehydroandrographolide;
14-dehydroxyandrographolide-12-sulfonic acid sodium salt;
14-alpha-lipoyl andrographolide;
andrographiside;
andrograpanin;
14-dehydroxy-11,12-didehydroandrographolide-3,19-bis(succinic acid) potassium salt;
14-dehydroxy-11,12-didehydroandrographolide-3,19-bis(succinic acid) potassium sodium salt;
(R)-((1R,5aS,6R,9aS)-1,5a-dimethyl-7-methylene-3-oxo-6-((E)-2-(2-oxo-2,5-dihydrofuran-3-yl)ethenyl)decahydro-1H-benzo [c]azapin-1-yl)methyl 2-amino-3-phenylpropanoate;
2-((1R,5R,6R,8aS)-6-hydroxy-5-(hydroxymethyl)-5,8a-dimethyl-2-methylenedecahydronaphthalen-1-yl)ethyl benzoate;
(1S,2R,4aS,5R,8aS)-1-formamido-1,4a-dimethyl-6-methylene-5-((E)-2-(2-oxo-2,5-dihydrofuran-3-yl)ethenyl)decahydronaphthalen-2-yl 5-((R)-1,2-pentanoate;
(1S,2R,4aS,5R,8aS)-1-formamido-1,4a-dimethyl-6-methylene-5-((E)-2-(2-oxo-2,5-dihydrofuran-3-yl)ethenyl)decahydronaphthalen-2-yl 2-nitrobenzoate; and
(S)-(1S,2R,4aS,5R,8aS)-1-formamido-1,4a-dimethyl-6-methylene-5-((E)-2-(2-oxo-2,5-dihydrofuran-3-yl)ethenyl)decahydronaphthalen-2-yl-2-amino-3-phenylpropanoate.

7. The antiviral combination according to claim 1, further comprising one or more additional therapeutic agents.

8. The antiviral combination according to claim 7, wherein the one or more additional therapeutic agents are antiviral agents.

9. The antiviral combination according to claim 8, wherein the one or more additional antiviral agents are selected from the group consisting of remdesivir, dexamethasone, gimsilumab, sarilumab, tocilizumab, anakinra, ruxolitinib, baricitinib, fedratinib, chloroquine, hydroxychloroquine, lopinavir, ritinovir, favipiravir, EIDD-2801, baricitinib, methylprednisolone, heparin, zinc, arbidiol/umifenovir, darunavir, oseltamivir, emtricitibine, tenofovir, baloxavir marboxil, danoprevir, dipyridamole, fingolimod, losartan, azithromycin, ribavirin, triazavirin, tranilast, ebastine, quercetin, glycyrrhizin, baicalin, patchouli alcohol, luteolin, hesperidin, emodin, kaempferol, lignin, betulinic acid, tanshinone, cryptotanshinone, dihydrotanshinone, I, tanshinone IIA, curcumin, shikonon and matrine.

10. A pharmaceutical composition comprising the antiviral combination according to claim 1 and at least one pharmaceutically acceptable carrier or diluent.

11. A pharmaceutical composition comprising an extract of Andrographis paniculata, and further comprising i) ursolic acid, or a pharmaceutically acceptable salt thereof, and piceid, or a derivative, or pharmaceutically acceptable salt thereof or ii) an extract of a plant selected from the group consisting of Eriobotrya japonica, rosemary and thyme, and/or an extract from Reynoutria japonica.

12. (canceled)

13. The pharmaceutical composition according to claim 10, wherein the composition is formulated for oral administration.

14. A method of treating or preventing a viral infection in a subject in need thereof comprising administering to the subject an effective amount of the antiviral combination according to claim 1.

15. The method according to claim 14, wherein the virus is selected from the group consisting of a picornavirus, a coronavirus, an influenza virus, a parainfluenza virus, a respiratory syncytial virus, an adenovirus, an enterovirus, and a metapneumovirus.

16. The method according to claim 14, wherein the viral infection is COVID-19.

17. A method of treating or preventing a bacterial infection in a subject in need thereof comprising administering to the subject an effective amount of the antiviral combination according to claim 1.

18. The method according to claim 17, wherein the bacterial infection is a secondary infection to a viral infection.

19.-26. (canceled)
